(19) United States
(12) Patent Application Publication
(10) Pub. No.: US 2003/0199757 A1
(43) Pub. Date: Oct. 23, 2003

(54) Method and apparatus of modulating radiation filtering during radiographic imaging

(76) Inventors: Thomas L. Toth, Brookfield, WI (US); Tsur Bernstein, Glendale, WI (US); Bruce M. Dunham, Mequon, WI (US)

Correspondence Address: ZIOLKOWSKI PATENT SOLUTIONS GROUP, LLC (GEMS), 14135 NORTH CEDARBURG ROAD, MEQUON, WI, 53097, US

(21) Appl. No.: 10/063,420

(22) Filed: Apr. 22, 2002

(51) Int. Cl. A61B 5/05; A61B 6/00

(52) U.S. Cl. 600/425

(57) ABSTRACT

The present invention includes a filtering apparatus for a CT imaging system or equivalently for an x-ray imaging system. The filtering apparatus is designed such that its attenuation profile may be changed prior to or during an imaging session. The attenuation profile can be modulated to mirror an attenuation pattern of a subject thereby optimizing radiation dose exposure to the subject. Furthermore, by implementing two opposing filters that are orthogonally oriented with respect to one another, the x-ray attenuation may be controlled along the x as well as z axis to shape the x-ray intensity.

### BACKGROUND OF THE INVENTION

The present invention relates generally to diagnostic imaging and, more particularly, to a method and apparatus of dynamically filtering radiation emitted toward a subject during radiographic imaging.

Typically, in radiographic imaging systems, an x-ray source emits x-rays toward a subject or object, such as a patient or a piece of luggage. Hereinafter, the terms “subject” and “object” may be interchangeably used to describe anything capable of being imaged. The beam, after being attenuated by the subject, impinges upon an array of radiation detectors. The intensity of the attenuated beam radiation received at the detector array is typically dependent upon the attenuation of the x-rays. Each detector element of the detector array produces a separate electrical signal indicative of the attenuated beam received by each detector element. The electrical signals are transmitted to a data processing system for analysis which ultimately produces an image.

In computed tomography (CT) imaging systems, the x-ray source and the detector array are rotated about a gantry within an imaging plane and around the subject. X-ray sources typically include x-ray tubes, which emit the x-rays as a beam at a focal point. X-ray detectors typically include a collimator for collimating x-ray beams received at the detector, a scintillator for converting x-rays to light energy adjacent the collimator, and a photodiode for receiving the light energy from an adjacent scintillator and producing electrical signals therefrom. Typically, each scintillator of a scintillator array converts x-rays to light energy. Each photodiode detects the light energy and generates a corresponding electrical signal. The outputs of the photodiodes are then transmitted to the data processing system for image reconstruction.

There is increasingly a need to reduce radiation dosage projected toward a patient during an imaging session. It is generally well known that significant dose reduction may be achieved by using a “bowtie” filter to shape the intensity profile of an x-ray beam. Surface dose reductions may be as much as 50% using a bowtie filter. It is also generally known that different anatomical regions of a patient may advantageously mandate different shaped bowtie filters to reduce radiation dosage. For example, scanning of the head or small region of a patient may require a bowtie filter shaped differently than a filter used during a large body scanning session. It is therefore desirable to have an imaging system with a large number of bowtie filter shapes available to best fit each patient. However, fashioning an imaging system with a sufficient number of bowtie filters to accommodate the idiosyncrasies encountered during scanning of numerous patients can be problematic in that each individual patient cannot be contemplated. Additionally, manufacturing an imaging system with a multitude of bowtie filters increases the overall manufacturing cost of the imaging system.

Therefore, it would be desirable to design an apparatus and method of dynamically filtering the radiation emitted toward the subject during imaging data acquisition with a single filter.

### BRIEF DESCRIPTION OF THE INVENTION

The present invention is a directed method and apparatus of dynamically filtering radiation projected toward a subject for data acquisition overcoming the aforementioned drawbacks.

The present invention includes a filtering apparatus for a CT imaging system or equivalently for an x-ray imaging system. The filtering apparatus is designed such that its shape may be changed prior to or during an imaging session. The shape of the filtering apparatus can be modulated to mirror an attenuation pattern of a subject thereby optimizing radiation dose exposure to the subject. Furthermore, by implementing two opposing filters that are orthogonally oriented with respect to one another, the x-ray attenuation may be controlled along the x as well as z axes to shape the x-ray intensity. A number of filtering apparatuses are contemplated.

In accordance with one aspect of the present invention, a method of diagnostic imaging comprises the steps of positioning a subject to be scanned into a scanning bay and projecting a radiation beam along a beam path toward the subject. The method further includes positioning a filter having an attenuation profile in the beam path. The attenuation profile of the filter is then modulated to define a desired attenuation profile. The method further includes acquiring diagnostic data of the subject and reconstructing an image of the subject from the diagnostic data.

In accordance with another aspect of the present invention, a method of acquiring diagnostic data of a subject comprises the steps of determining an attenuation pattern for acquiring diagnostic data of a subject to be scanned and presetting a first filter to a desired attenuation profile. The method further includes the step of projecting high frequency electromagnetic energy toward the subject to acquire diagnostic data of the subject. During the projection of high frequency electromagnetic energy, a second filter having an attenuation profile is translated such that the attenuation profiles of the first filter and the second filter is a function of the attenuation pattern of the subject.

In accordance with a further aspect of the present invention, a method of diagnostic imaging includes the steps of positioning a subject to be scanned on a table in a scanning bay and projecting high frequency electromagnetic energy toward the subject. The method further includes dynamically filtering the high frequency electromagnetic energy with at least one filter and acquiring imaging data of the subject. A set of images of the subject from the imaging data are then reconstructed. With the subject removed from the scanning bay, high frequency electromagnetic energy is again projected toward the detector absent the subject and table and dynamically filtered with the at least one filter. The method further includes acquiring scan data attributable to the at least one filter and generating a set of calibration data attributable to the at least one filter to be used in reconstructing artifact free images of the subject.

In accordance with yet another aspect of the present invention, a radiation emitting system comprises a scanning bay configured to position the subject to be scanned in a path of radiation as well as a radiation projection source configured to project radiation toward the subject. The system further includes a radiation filter having a variable attenuation profile. A computer is also provided and programmed to determine an attenuation pattern of the subject and modulate the variable attenuation profile of the radiation filter as a function of the attenuation pattern of the subject.

In accordance with a further aspect of the present invention, a radiation emitting imaging system is provided. The imaging system includes a scanning bay and a moveable table configured to move a subject to be scanned fore and aft along a first direction within the scanning bay. The system further includes an x-ray projection source configured to project x-rays toward the subject. A first attenuator is provided and configured to attenuate x-rays along a first axis. A second attenuator is also provided and configured to attenuate x-rays along a second axis. Both the first attenuator and second attenuator are translatable in the first direction. The imaging system further includes a computer programmed to calibrate the first attenuator to have a desired attenuation profile and calibrate the second attenuator to have a desired attenuation profile. The computer is further programmed to move the subject along the first direction and simultaneously therewith, translate at least one of the first attenuator and the second attenuator in the first direction.

In accordance with yet another aspect of the present invention, a computer readable storage medium is provided and has stored thereon a computer program representing a set of instructions that when executed by a computer causes the computer to move a subject to be scanned into a scan position. The set of instructions further causes the computer to determine an attenuation pattern of the subject and manipulate an attenuation profile of a filter configured to filter x-rays projected toward a subject. The computer is also instructed to acquire imaging data of the subject and reconstruct at least one image therefrom.

In accordance with another aspect of the present invention, a filtering apparatus to filter radiation projected toward a subject to be scanned is provided. The filtering apparatus includes a body having a plurality of hollow tubes parallelly arranged and configured to receive and discharge attenuating fluid to define an attenuation profile as a function of an attenuation pattern of the subject.

In accordance with a further aspect of the present invention, a filtering apparatus to filter radiation projected toward a subject to be scanned includes a body constructed so as to be capable of having a plurality of attenuating rods. Each of the attenuating rods is placeable in the body such that an attenuation profile as a function of an attenuation pattern of the subject is defined.

In accordance with yet another aspect of the present invention, a filtering apparatus to filter radiation projected toward a subject to be scanned comprises a flexible bladder containing attenuating fluid. The flexible bladder is configured to be manipulated to modulate the attenuating fluid such that an attenuation profile as a function of an attenuation pattern of the subject is defined.

Various other features, objects and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a pictorial view of a CT imaging system.

FIG. 2 is a block schematic diagram of the system illustrated in FIG. 1.

FIG. 3 is a plan view of a representative x-ray system.

FIG. 4 is a sectional view of a portion of the x-ray system shown in FIG. 1.

FIG. 5 is a perspective view of one embodiment of a dynamic filter in accordance with the present invention.

FIG. 6 is a perspective view of another embodiment of a dynamic filter in accordance with the present invention.

FIG. 7 is a perspective view of another embodiment of a dynamic filter in accordance with the present invention.

FIG. 8 is a perspective view of another embodiment of a dynamic filter in accordance with the present invention.

FIG. 9 is a representation of a filtering apparatus during translation in accordance with another aspect of the present invention.

### DETAILED DESCRIPTION

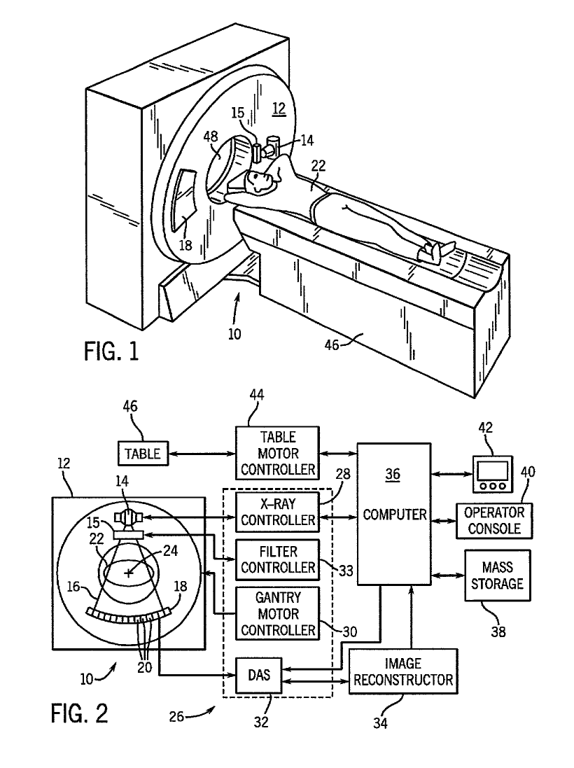

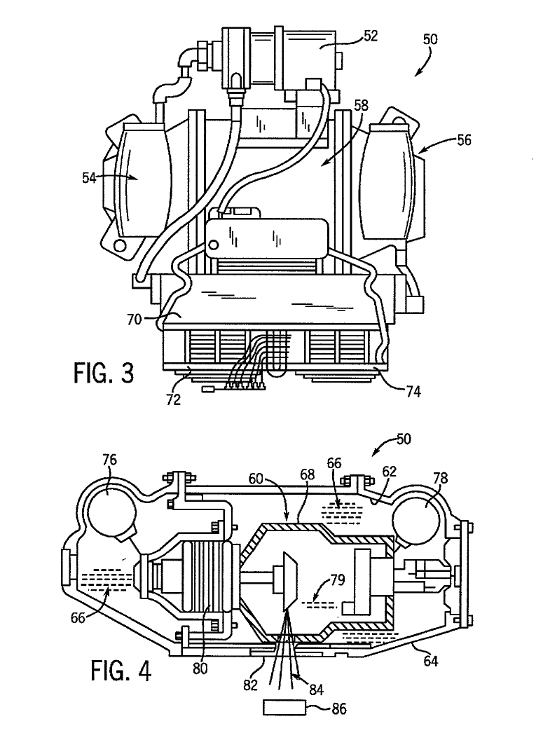

The present invention is described with respect to a radiographic imaging system such as the CT system shown in FIGS. 1-2 and the x-ray system shown in FIGS. 3-4. However, it will be appreciated by those skilled in the art that the present invention is equally applicable for use with other radiographic imaging systems. Moreover, the present invention will be described with respect to the emission and detection of x-rays. However, one skilled in the art will further appreciate, that the present invention is equally applicable for the emission and detection of other high frequency electromagnetic energy.

Referring to FIGS. 1 and 2, a “third generation” CT imaging system 10 is shown as including a gantry 12. The present invention, however, is applicable with other CT systems. Gantry 12 has an x-ray source 14 that projects a beam of x-rays 16 through filter 15 toward a detector array 18 on the opposite side of the gantry 12. Detector array 18 is formed by a plurality of detectors 20 which together sense the projected x-rays that pass through a medical patient 22. Each detector 20 produces an electrical signal that represents the intensity of an impinging x-ray beam and hence the attenuated beam as it passes through the patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to an x-ray source 14, a gantry motor controller 30 that controls the rotational speed and position of gantry 12, and filter controller 33 that controls filter 15. A data acquisition system (DAS) 32 in control mechanism 26 samples analog data from detectors 20 and converts the data to digital signals for subsequent processing. An image reconstructor 34 receives sampled and digitized x-ray data from DAS 32 and performs high speed reconstruction. The reconstructed image is applied as an input to a computer 36 which stores the image in a mass storage device 38.

Computer 36 also receives commands and scanning parameters from an operator via console 40 that has a keyboard. An associated cathode ray tube display 42 allows the operator to observe the reconstructed image and other data from computer 36. The operator supplied commands and parameters are used by computer 36 to provide control signals and information to DAS 32, x-ray controller 28 and gantry motor controller 30. In addition, computer 36 operates a table motor controller 44 which controls a motorized table 46 to position patient 22 and gantry 12. Particularly, table 46 moves portions of patient 22 through a gantry opening 48.

Referring now to FIGS. 3-4, an x-ray system 50 incorporating the present invention is shown. The x-ray system 50 includes an oil pump 52, an anode end 54, and a cathode end 56. A central enclosure 58 is provided and positioned between the anode end 54 and the cathode end 56. Housed within the central enclosure 58 is an x-ray generating device or x-ray tube 60. A fluid chamber 62 is provided and housed within a lead lined casing 64. Fluid chamber 62 is typically filled with coolant 66 that will be used to dissipate heat within the x-ray generating device 60. Coolant 66 is typically a dielectric oil, but other coolants including air may be implemented. Oil pump 52 circulates the coolant through the x-ray system 50 to cool the x-ray generating device 60 and to insulate casing 64 from high electrical charges found within vacuum vessel 68. To cool the coolant to proper temperatures, a radiator 70 is provided and positioned at one side of the central enclosure 58. Additionally, fans 72, 74 may be mounted near the radiator 70 to provide cooling air flow over the radiator 70 as the dielectric oil circulates therethrough. Electrical connections are provided in anode receptacle 76 and cathode receptacle 78 that allow electrons 79 to flow through the x-ray system 50.

Casing 64 is typically formed of an aluminum-based material and lined with lead to prevent stray x-ray emissions. A stator 70 is also provided adjacent to vacuum vessel 68 and within the casing 64. A window 82 is provided that allows for x-ray emissions created within the system 50 to exit the system and be projected toward an object, such as, a medical patient for diagnostic imaging. Typically, window 82 is formed in casing 64. Casing 64 is designed such that most generated x-rays 84 are blocked from emission except through window 82.

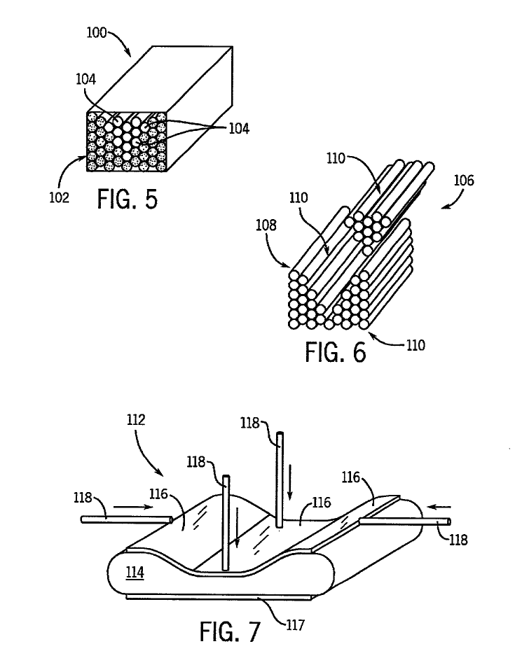

Referring now to FIGS. 5-9, a number of filter embodiments will be described. It should be noted that each of the embodiments described may be implemented as a pre-patient bowtie filter in a CT imaging system similar to filter 15 shown in FIGS. 1-2 or as a pre-patient filter 86 for an x-ray system similar to that shown in FIGS. 3-4. Specifically, a number of filter embodiments will be described wherein each of the filters may be modulated or “morphed” to define a desired attenuation profile specific to the particular imaging needs of an imaging session. For example, the attenuation profile of the filter may be modulated such that radiation exposure to particular organs is reduced without sacrificing or jeopardizing radiation exposure to other particular regions of interest. As a result, organs or regions of interest either sensitive to radiation exposure or not subject of the imaging session are not unnecessarily subjected to radiation exposure. Additionally, the attenuation profile of the filter may be modulated as a function of viewing angle. For example, the attenuation profile of the filter may be manipulated to filter radiation for a wider region of interest for a top view data acquisition position and likewise be manipulated to have a more narrow profile for a side view data acquisition position. The attenuation profile of the filter may also be modulated as a function of filter position along an imaging axis. For example, the attenuation profile of the filter may be dynamically manipulated during translation of the subject and/or filter to reduce radiation exposure in dose avoidance or reduction regions located between regions of interest. “Dose avoidance” and “dose reduction” refers to certain organs or anatomical regions where reduced radiation exposure is desired during an imaging session. While complete blockage of radiation to these areas is desired, reducing but not eliminating radiation exposure to these regions is acceptable. Therefore, it remains desirable to develop an attenuation profile that reduces if not eliminates radiation exposure to certain anatomical regions of the subject but SNR may be sacrificed with respect to these “avoidance” or “reduction” regions.

Referring now to FIG. 5, one embodiment of the present invention is shown. In this embodiment, filter 100 includes a body 102 defined by a plurality of hollow tubes 104. Hollow tubes 104 are configured to receive attenuating fluid such as a contrast agent. As shown, a selected number of the hollow tubes have been flooded with the attenuating fluid to define an attenuation profile. The attenuation profile defined by the attenuating fluid flooded into the hollow tubes is only one example. That is, any number of the hollow tubes may be filled with attenuating fluid to define a desired attenuation profile. The attenuating fluid is stored in a reservoir (not shown) and a computer or control mechanism floods the tubes to define the desired attenuation profile needed for the imaging session or for a moment in the imaging session. That is, depending upon the needs of the imaging session, the tubes may be filled and flushed dynamically throughout the imaging session to vary the attenuation profile during data acquisition. A number of techniques of removing or flushing attenuating fluid from a tube are contemplated including a computer controlled system of valves (not shown) that apply compressed gas to the chambers. Alternately, a series of honeycombed cavities may be equivalently implemented in place of the hollow tubes.

Referring now to FIG. 6, another embodiment of the filter in accordance with the present invention is shown. In this embodiment, filter 106 includes a body 108 defined by a number of attenuating rods 110. Operation of filter 106 is similar to operation of filter 100 of FIG. 5. With filter 106, each attenuating rod 110 is positioned within the body such that the plurality of attenuating rods as a whole defines the desired attenuation profile. Filter 106 may be used to filter radiation in a couple of ways. First, that portion of the plurality of attenuating rods 110 having attenuating rods removed may be placed in the x-ray beam path or, conversely, the attenuating rods 110 disposed from the rest of the attenuating rods may be slid into the x-ray beam path. A control and/or computer may be programmed to reposition the attenuating rods to define the desired attenuation profile.

Referring now to FIG. 7, another preferred embodiment of a filtering apparatus 112 includes a flexible bladder 114 containing attenuating fluid positioned between an upper plate 116 and a lower plate or base 117. Bladder 14 is sufficiently flexible such that the attenuating fluid contained therein may be modulated or manipulated to define the desired attenuation profile. Bladder 114 may contain attenuating liquid, gelatin, beads, or the like. Upper plate 16 is fabricated from a flexible x-ray transparent material such as plastic that, in response to an applied force, alters the shape of the flexible bladder 114. In one embodiment, the upper plate responds to a force applied by at least one of a number of moveable rods 118. The moveable rods 118 are controlled by a computer to distort the upper plate such that the flexible bladder is likewise distorted. Base plate 118 supports the flexible bladder and is fabricated from a solid x-ray transparent material. Alternatively, base plate 117 could be fabricated to contain x-ray spectral filtration material. It should be noted that flexible bladder 114, upper plate 116, and base plate 117 are each fabricated from an x-ray transparent material so that x-rays are attenuated primarily by the attenuating fluid rather than the bladder or plates.

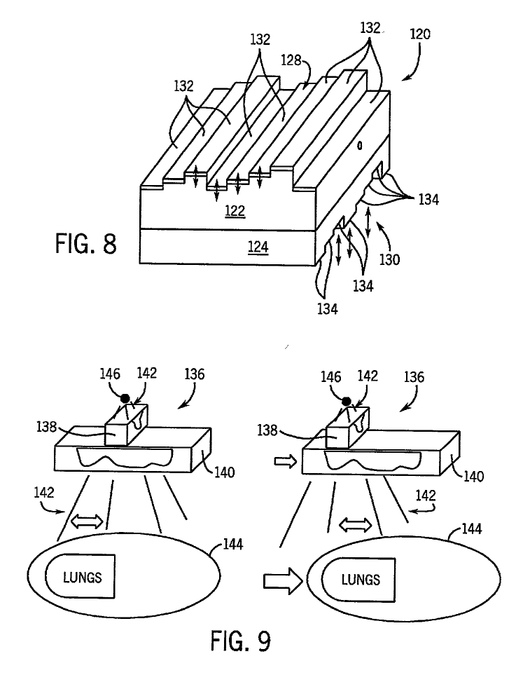

Referring now to FIG. 8, another embodiment of a filtering apparatus in accordance with the present invention is shown. In this embodiment, filter 120 includes a first bladder 122 and a second bladder 124. Each bladder 122, 124 is designed to contain attenuating fluid such as attenuating liquid, gelatin, or beads. Filter 120 further includes an intermediary plate 126 disposed between bladder 122 and bladder 124. Filter 120 further includes an upper plate 128 and a lower plate 130. Each plate 128, 130 is formed from a plurality of parallelly aligned slots 132, 134. The slots 132 and 134 of each plate 128 and 130, respectively, impart or release a force applied to bladders 122 and 124. That is, each slot 132 of plate 128 moves perpendicularly with respect to plate 126 to impart a desired force onto bladder 122 such that the attenuating fluid contained within bladder 122 defines a desired attenuation profile. Slots 134 of plate 130 operate in a similar fashion to define a desired attenuation profile for bladder 124. For example, slots 132 may be moved by a computer controlled mechanism such as step actuators to impart a force on bladder 122 to define an attenuation profile along an x axis whereas slots 130 of plate 134 respond to another set of step actuators to define an attenuation profile along a z axis. Collectively, slots 132 and 134 cooperatively define a desired attenuation profile that mirrors a dual-axes attenuation pattern of the subject. The attenuation pattern of the subject may be determined from a scout scan of the subject. Additionally, filter 120 may be implemented with only one of the bladders 122, 124 and only one of the plates 128-130 of slots 132, 134. In this alternate single bladder embodiment, an attenuation profile is defined only along one axis. Moreover, in accordance with another embodiment, the flexible bladders 122, 124 may be manipulated by step actuators (not shown) directly without plates 128 and 130.

Shown in FIG. 9 is a representation of a filtering apparatus in accordance with another aspect of the present invention during translation in a first direction. In this embodiment, filtering apparatus 136 comprises an x axis filter 138 and a z axis filter 140. Filtering apparatus 136 is designed to filter x-ray beams 142 projected toward a subject 144 by an x-ray source 146. Filters 138 and 140 may comprise any one of the dynamic filters described with respect of FIGS. 5-8. Accordingly, an attenuation profile of filter 138 and an attenuation profile of filter 140 are defined for a moment of x-ray projection. Preferably, the attenuation profiles are defined prior to the imaging session based on the attenuation pattern of the subject 144 determined from a scout scan, but, alternately, the attenuation profiles may be defined during x-ray projection or from a data base of patient demographic information. As shown in FIG. 9, the attenuation profile of filter 138 is set as is the attenuation profile of filter 140. Collectively, attenuation profiles will mirror the attenuation patterns of the subject 144 in both the x and z axis. In operation, as the subject 144 is translated in a first direction by a moveable table filter 138 is synchronously translated in the first direction as well. As a result, the collective attenuation profile of filters 138 and 140 mirror the attenuation pattern of the subject 144 during translation of the patient in the first direction along the z axis. As such, the dosage applied to various anatomical regions of the patient may be optimized to eliminate over exposure of radiation to the patient. While FIG. 9 shows translation of the z axis filter 140, the x axis filter 138 could likewise be translated with patient movement.

As is indicated previously, a scout scan may be performed of the subject to determine a filter contour that best fits the complement of the patient's attenuation pattern. Accordingly, special needs of the imaging session for the patient such as dose avoidance or reduction regions or regions of increased x-ray necessity may be accounted for in defining the patient's attenuation pattern. Also, as indicated previously, the attenuation profile of filters may be preset prior to the imaging session or dynamically modulated during the imaging session to mirror or complement the attenuation pattern of the subject.

In a further embodiment of the present invention, one or more dynamic filters may be used to filter radiation during the acquisition of imaging data of a subject. A set of images can then be reconstructed according to well known reconstruction techniques of the subject based on the filtered imaging data. However, the imaging data is susceptible to the presence of artifacts and the set of images associated with the one or more filters itself. Accordingly, the patient is removed from the scanning bay and another set of scan data is acquired wherein the one or more filters are dynamically defined as they were during the imaging of the patient. As a result, a set of calibration data is obtained attributable to the one or more dynamically configured filters. Therefore, a set of images of the of the patient can be reconstructed using the calibration data and usual correction methods. The present invention has been described with respect to a number of embodiments of a dynamic filter to be implemented in a radiographic imaging system. The various embodiments may be utilized to dynamically modulate the attenuation profile of the filter prior to and/or during the imaging session to mirror the attenuation pattern of the subject and thereby reduce radiation exposure to the patient.

Accordingly, in accordance with one embodiment of the present invention, a method of diagnostic imaging comprises the steps of positioning a subject to be scanned into a scanning bay and projecting a radiation beam along a beam path toward the subject. The method further includes positioning a filter having an attenuation profile in the beam path. The attenuation profile of the filter is then modulated to define a desired attenuation profile. The method further includes acquiring diagnostic data of the subject and reconstructing an image of the subject from the diagnostic data.

In accordance with another embodiment of the present invention, a method of acquiring diagnostic data of a subject comprises the steps of determining an attenuation pattern for acquiring diagnostic data of a subject to be scanned and presetting a first filter to a desired attenuation profile. The method further includes the step of projecting high frequency electromagnetic energy toward the subject to acquire diagnostic data of the subject. During the projection of high frequency electromagnetic energy, a second filter having an attenuation profile is translated such that the attenuation profiles of the first filter and the second filter is a function of the attenuation pattern of the subject.

In accordance with a further embodiment of the present invention, a method of diagnostic imaging includes the steps of positioning a subject to be scanned on a table in a scanning bay and projecting high frequency electromagnetic energy toward the subject. The method further includes dynamically filtering the high frequency electromagnetic energy with at least one filter and acquiring imaging data of the subject. A set of images of the subject from the imaging data are then reconstructed. With the subject removed from the scanning bay, high frequency electromagnetic energy is again projected toward the detector absent the subject and table and dynamically filtered with the at least one filter. As a result, a set of calibration data is obtained attributable to the one or more dynamically configured filters. Therefore, a set of images of the patient can be reconstructed using the calibration data and usual correction methods.

In accordance with yet another embodiment of the present invention, a radiation emitting system comprises a scanning bay configured to position the subject to be scanned in a path of radiation as well as a radiation projection source configured to project radiation toward the subject. The system further includes a radiation filter having a variable attenuation profile. A computer is also provided and programmed to determine an attenuation pattern of the subject and modulate the variable attenuation profile of the radiation filter as a function of the attenuation pattern of the subject.

In accordance with a further embodiment of the present invention, a radiation emitting imaging system is provided. The imaging system includes a scanning bay and a moveable table configured to move a subject to be scanned fore and aft along a first direction within the scanning bay. The system further includes an x-ray projection source configured to project x-rays toward the subject. A first attenuator is provided and configured to attenuate x-rays along a first axis. A second attenuator is also provided and configured to attenuate x-rays along a second axis. Both the first attenuator and second attenuator are translatable in the first direction. The imaging system further includes a computer programmed to calibrate the first attenuator to have a desired attenuation profile and calibrate the second attenuator to have a desired attenuation profile. The computer is further programmed to move the subject along the first direction and simultaneously therewith, translate at least one of the first attenuator and the second attenuator in the first direction.

In accordance with yet another embodiment of the present invention, a computer readable storage medium is provided and has stored thereon a computer program representing a set of instructions that when executed by a computer causes the computer to move a subject to be scanned into a scan position. The set of instructions further causes the computer to determine an attenuation pattern of the subject and manipulate an attenuation profile of a filter configured to filter x-rays projected toward a subject. The computer is also instructed to acquire imaging data of the subject and reconstruct at least one image therefrom.

In accordance with another embodiment of the present invention, a filtering apparatus to filter radiation projected toward a subject to be scanned is provided. The filtering apparatus includes a body having a plurality of hollow tubes parallelly arranged and configured to receive and discharge attenuating fluid to define an attenuation profile as a function of an attenuation pattern of the subject.

In accordance with a further embodiment of the present invention, a filtering apparatus to filter radiation projected toward a subject to be scanned includes a body constructed to be capable of having a plurality of attenuating rods. Each of the attenuating rods is placeable in the body such that an attenuation profile as function of an attenuation pattern of the subject is defined.

In accordance with yet another embodiment of the present invention, a filtering apparatus to filter radiation projected toward a subject to be scanned comprises a flexible bladder containing attenuating fluid. The flexible bladder is configured to be manipulated to modulate the attenuating fluid such that an attenuation profile as a function of an attenuation pattern of the subject is defined.

The present invention has been described in terms of the preferred embodiment, and it is recognized that equivalents, alternatives, and modifications, aside from those expressly stated, are possible and within the scope of the appending claims.

What is claimed is:

1. A method of diagnostic imaging comprising the steps of:
positioning a subject to be scanned into a scanning bay;
projecting a radiation beam along a beam path toward the subject;
positioning a filter having an attenuation profile in the beam path;
modulating the attenuation profile to define a desired attenuation profile;
acquiring diagnostic data of the subject; and
reconstructing an image of the subject from the diagnostic data.

2. The method of claim 1 further comprising the step of modulating the attenuation profile to the desired attenuation profile to reduce radiation exposure to one or more regions of the subject.

3. The method of claim 2 further comprising the step of protecting specific anatomical regions of the subject against substantial radiation exposure.

4. The method of claim 1 further comprising the step of modulating the attenuation profile to the desired attenuation profile as a function of viewing angle.

5. The method of claim 1 wherein the filter includes a body having a number of hollow tubes and wherein the step of modulating further includes the step of filling a selected number of the hollow tubes with attenuating material to define the desired attenuation profile.

6. The method of claim 5 wherein the attenuating material includes liquid attenuator.

7. The method of claim 1 wherein the filter includes a body having a plurality of removable attenuating rods and wherein the step of modulating further includes the step of positioning a number of the removable attenuating rods in the body to define the desired attenuation profile.

8. The method of claim 1 wherein the filter includes a flexible bladder having a shape and containing attenuating material and wherein the step of modulating further includes the step of altering the shape of the flexible bladder to define the desired attenuation profile.

9. The method of claim 8 wherein the step of altering further includes the step of applying pressure to the flexible bladder.

10. The method of claim 9 wherein the filter includes a solid x-ray transparent base plate supportive of the flexible bladder and an upper plate of flexible x-ray transparent plastic positioned adjacently atop the flexible bladder and wherein the step of applying pressure further includes the step of distorting the upper plate.

11. The method of claim 10 wherein the step of distorting includes the step of applying force to one or more region of the upper plate with one or more movable rods.

12. The method of claim 10 wherein the upper plate includes a plurality of parallel slots and wherein the step of distorting includes the step of positioning a number of the parallel slots to either one of apply force to the flexible bladder or reduce force applied to the flexible bladder to define the desired attenuation profile.

13. The method of claim 1 further comprising the step of modulating the attenuation profile of the filter during the acquiring of diagnostic data.

14. The method of claim 1 further comprising the step of performing a scout scan to determine a patient attenuation pattern and defining the desired attenuation profile of the filter as a function of the patient attenuation pattern.

15. A method of acquiring diagnostic data of a subject comprising the steps of:
determining an attenuation pattern for acquiring diagnostic data of a subject to be scanned;
presetting a first filter to a desired attenuation profile;
projecting HF electromagnetic energy toward the subject to acquire diagnostic data of the subject;
during the projecting, translating a second filter having an attenuation profile such that the attenuation profiles of the first filter and the second filter is a function of the attenuation pattern of the subject.

16. The method of claim 15 wherein the step of determining an attenuation pattern further comprises the step of initiating a scout scan of the subject.

17. The method of claim 16 wherein the step of presetting the first filter further comprises the step of determining a filter contour that complements the attenuation pattern of the subject.

18. The method of claim 17 wherein the step of determining the filter contour further comprises the step of accounting for at least one of dose reduction regions of the subject and regions of the subject where increased HF electromagnetic energy is desired.

19. The method of claim 15 wherein the first filter includes an x axis filter and the second filter includes a z axis filter.

20. The method of claim 15 wherein the step of translating further comprises the step of moving the second filter synchronically with movement of the subject.

21. A method of diagnostic imaging comprising the steps:
positioning a subject to be scanned on a table in a scanning bay;
projecting HF electromagnetic energy toward the subject and a detector assembly;
dynamically filtering the HF electromagnetic energy with at least one filter;
acquiring imaging data of the subject;
reconstructing a set of images of the subject from the imaging data;
removing the subject and table from the scanning bay;
projecting HF electromagnetic energy toward the detector assembly and dynamically filtering HF electromagnetic energy with the at least one filter;
acquiring data attributable to the at least one filter;
generating a set of images attributable to the at least one filter; and
recalibrating the at least one filter such that images absent artifacts attributable to the at least one filter are absent from reconstructed images of the subject.

22. The method of claim 21 further comprising the step of determining a filter calibration sequence and reacquiring imaging data of the subject with the HF electromagnetic energy being filtered by the at least one filter wherein the at least one filter filters HF electromagnetic energy according to the filter calibration sequence.

23. The method of claim 22 wherein the at least one filter has an attenuation profile and further comprising the step of modulating the attenuation profile during the step of filtering based on the calibration sequence.

24. The method of claim 21 further comprising the step of reconstructing a final set of images of the subject having the artifacts attributable to the at least one filter removed.

25. A radiation emitting imaging system comprising:
a scanning bay configured to position a subject to be scanned in a path of radiation;
a radiation projection source configured to project radiation toward the subject;
a radiation filter having a variable attenuation profile; and
a computer programmed to:
determine an attenuation pattern of the subject; and
modulate the variable attenuation profile of the radiation filter as a function of the attenuation pattern of the subject.

26. The radiation emitting imaging system of claim 25 wherein the computer is further programmed to modulate the variable attenuation profile of the radiation filter during radiation projection toward the subject.

27. The radiation emitting imaging system of claim 25 wherein the computer is further programmed to determine does reduction regions of the subject and further programmed to modulate the variable attenuation profile such that radiation exposure to the dose reduction regions is reduced.

28. The radiation emitting imaging system of claim 27 wherein the dose reduction regions include anatomical regions not to be imaged.

29. The radiation emitting imaging system of claim 25 wherein the computer is further programmed to modulate the variable attenuation pattern as a function of viewing angle.

30. The radiation emitting imaging system of claim 25 wherein the radiation filter includes a body of fillable hollow tubes and wherein the computer is further programmed to flood the hollow tubes with attenuating fluid to mirror the attenuation pattern of the subject.

31. The radiation emitting imaging system of claim 25 wherein the radiation filter includes a body of attenuating rods and wherein the computer is further programmed to manipulate the attenuating rods to mirror the attenuation pattern of the subject.

32. The radiation emitting imaging system of claim 25 wherein the radiation filter includes a body having an upper plate, a lower plate, a flexible bladder containing attenuating fluid disposed between the upper plate and the lower plate and wherein the computer is further programmed to modulate at least one of the upper plate and the lower plate to manipulate the attenuating fluid contained within the flexible bladder to mirror the attenuation pattern of the subject.

33. The radiation emitting imaging system of claim 32 wherein the upper plate includes a plurality of parallelly aligned slots and wherein the computer is further programmed to modulate the plurality of parallelly aligned slots to manipulate the attenuating fluid contained within the flexible bladder to mirror the attenuation pattern of the subject.

34. The radiation emitting imaging system of claim 25 wherein the computer is further programmed to initiate a scout scan of the subject and determine the attenuation pattern of the subject therefrom.

35. The radiation emitting imaging system of claim 25 incorporated into a CT system.

36. A radiation emitting imaging system comprising:
a scanning bay;
a movable table configured to move a subject to be scanned fore and aft along a first direction within the scanning bay;
an x-ray projection source configured to project x-rays toward the subject;
a first attenuator configured to attenuate x-rays along a first axis and translatable in the first direction;
a second attenuator configured to attenuate x-rays along a second axis and translatable in the first direction;
a computer programmed to:
calibrate the first attenuator to have a desired attenuation profile;
calibrate the second attenuator to have a desired attenuation profile;
move the subject along the first direction;
simultaneously therewith, translate at least one of the first attenuator and the second attenuator in the first direction.

37. The radiation emitting imaging system of claim 36 wherein the computer is further programmed to determine an attenuation pattern of the subject and calibrate the attenuation profiles of the first attenuator and the second attenuator as a function of the attenuation pattern of the subject during translation of at least one of the first attenuator and the second attenuator in the first direction.

38. The radiation emitting imaging system of claim 37 where the computer is further programmed to determine the attenuation pattern of the subject from a scout scan.

39. The radiation emitting imaging system of claim 36 wherein the computer is further programmed to determine dose reduction regions of the subject and further programmed to modulate the variable attenuation profile such that radiation exposure to the dose reduction regions is reduced.

40. The radiation emitting imaging system of claim 39 wherein the computer is further programmed to modulate the variable attenuation pattern as a function of viewing angle.

41. A computer readable storage medium having stored thereon a computer program and representing a set of instructions that when executed by a computer causes the computer to:
move a subject to be scanned into a scan position;
determine an attenuation pattern of the subject;
manipulate an attenuation profile of a filter configured to filter x-rays projected toward the subject; and
acquire imaging data of the subject and reconstruct at least one image therefrom.

42. The computer readable storage medium of claim 41 wherein the set of instructions further causes the computer to manipulate the attenuation profile of the filter during x-ray projection.

43. The computer readable storage medium of claim 41 wherein the set of instructions further causes the computer to manipulate the attenuation pattern and reduce x-ray exposure to dose reduction regions of the subject.

44. The computer readable storage medium of claim 43 wherein the set of instructions further causes the computer to modulate the variable attenuation pattern as a function of viewing angle.

45. The computer readable storage medium of claim 41 wherein the filter includes a body of fillable hollow tubes and wherein the computer is further programmed to flood the hollow tubes with attenuating fluid to mirror the attenuation pattern of the subject.

46. The computer readable storage medium of claim 41 wherein the filter includes a body of attenuating rods and wherein the computer is further programmed to manipulate the attenuating rods as a function of the attenuation pattern of the subject.

47. The computer readable storage medium of claim 41 wherein the filter includes a body having an upper plate, a lower plate, a flexible bladder containing attenuating fluid disposed between the upper plate and the lower plate and wherein the computer is further programmed to modulate at least one of the upper plate and the lower plate to manipulate the attenuating fluid contained within the flexible bladder as a function of the attenuation pattern of the subject.

48. The computer readable storage medium of claim 47 wherein the filter includes a plurality of parallelly aligned slots and wherein the computer is further programmed to modulate the plurality of parallelly aligned slots to manipulate the attenuating fluid contained within the flexible bladder as a function of the attenuation pattern of the subject.

49. A filtering apparatus to filter radiation projected toward a subject to be scanned, the filtering apparatus comprising a body having a plurality of hollow tubes parallelly arranged and configured to receive and discharge attenuating fluid to define an attenuation profile as a function of an attenuation pattern of the subject.

50. A filtering apparatus to filter radiation projected toward a subject to be scanned, the filtering apparatus comprising a body constructed so as to be capable of having a plurality of attenuating rods therein, wherein each of the plurality of attenuating rods is placeable in the body such that an attenuation profile is defined as a function of an attenuation pattern of the subject.

51. A filtering apparatus to filter radiation projected toward a subject to be scanned, the filtering apparatus comprising a flexible bladder containing attenuating fluid wherein the flexible bladder is manipulated to modulate the attenuating fluid such that an attenuation profile as a function of an attenuation pattern of the subject is defined.

52. The filtering apparatus of claim 51 further comprising:
a first plate positioned adjacent one side of the flexible bladder;
a second plate positioned adjacent another side of the flexible bladder; and
wherein at least one of the first plate and the second plate is configured to respond to an applied force to manipulate the flexible bladder to modulate the attenuating fluid such that the attenuation profile is defined.

53. The filtering apparatus of claim 52 wherein the first plate includes a number of parallelly aligned slots configured to impart a force on the flexible bladder.

54. The filtering apparatus of claim 52 further comprising at least one distortion rod configured to provide the applied force to one of the first plate and the second plate.

55. The filtering apparatus of claim 52 wherein the first plate comprises a flexible x-ray transparent plastic material and the second plate comprises an inflexible x-ray transparent material.
